(19) United States
(12) Patent Application Publication
(10) Pub. No.: US 2023/0165581 A1
(43) Pub. Date: Jun. 1, 2023

(54) SUTURE DEVICE, TREATMENT DEVICE WITH SUTURE DEVICE, AND TREATMENT SYSTEM

(71) Applicants: Ping WANG, Hangzhou, Zhejiang Province (CN); ZHEJIANG ZUOYUAN MEDICAL TECHNOLOGY CO., LTD., Hangzhou, Zhejiang Province (CN)

(72) Inventors: Ping WAN, Hangzhou, Zhejiang Province (CN); Zhaoteng ZHOU, Hangzhou, Zhejiang Province (CN); Rou WAN, Hangzhou, Zhejiang Province (CN)

(21) Appl. No.: 17/921,791

(22) PCT Filed: Apr. 27, 2020
(86) PCT No.: PCT/CN2020/087329

§ 371 Date: Oct. 27, 2022

(51) Int. Cl. A61B 17/04; A61B 17/062; A61B 17/3205

(52) U.S. Cl. CPC A61B 17/0469; A61B 17/062; A61B 17/32056; A61B 2017/00477

(57) ABSTRACT

The invention provides a suture device, a treatment device with the suture device and a treatment system. The treatment system includes an endoscope and the treatment system. The treatment system is used in cooperation with the endoscope and includes an operating handle and the suture device. The suture device comprises a main shell, a suture needle, a needle feeding assembly and a needle anti-backoff assembly. One end of the suture needle is connected to a suture line, and the other end is a tip. The needle feeding assembly is arranged on the main shell and forms a separable engagement relationship with the suture needle so as to control advancing of the suture needle. The needle anti-backoff assembly is arranged on the main shell and forms a separable engagement relationship with the suture needle so as to prevent the withdrawal of the suture needle.

### TECHNICAL FIELD

The present invention relates to a treatment system, and more particularly, to a suture device, a treatment device having the suture device, and a treatment system.

### BACKGROUND

With advances in science and technology, surgery has been developed from traditional open procedures, minimally invasive procedures to non-invasive procedures. Non-invasive surgery enters the surgical area through the natural orifice (such as the esophagus, the rectum, the uterus, etc.) of the human body, has no external wounds and little bleeding, not only reduces the physiological and psychological trauma of the patient, but also reduces the anesthesia depth, thereby greatly reducing the risk of anesthesia. Non-invasive procedures can effectively reduce post-operative complications and are more beneficial for higher-risk patients.

Since non-invasive surgery has only begun in recent years, the corresponding equipment is relatively scarce. Especially most medical equipment cannot achieve the suture of surgery in vivo. U.S. Pat. No. 7,344,545 discloses an endoscopic suturing system for a surgical procedure that operates in vitro and suture in vivo for the purpose of treatment. The suturing system includes a component having first and second arms and a needle recovery member that needs to be rigidly aligned with a looped arm. The arrangement of tissue grasping arms and the needle recovery member makes the system bulky, making it difficult for practical applications in endoscopic procedures.

An endoscopic suturing system is also disclosed by Apollo Endosurgery Inc. The suturing device can be inserted into the body through the natural orifice of the human body in conjunction with an endoscope or other steerable guide device. The system is complex in structure, numerous in equipment parts, complex in manufacturing and high in cost. When the system is in use, there is a need to occupy three insertion tubes at the same time. Since there are only three insertion tubes, the operating device needs to be exchanged in one insertion tube (i.e., the device previously occupied the insertion tube needs to be removed at first and then a new device is inserted into the insertion tube), and the suturing steps are cumbersome.

In particular, when the suturing device provided by the Apollo Endosurgery Inc. sutures wound tissue in vivo, the tissue is hooked after the tissue is punctured by a special spin-drilling and hooking device, and then the suture needle can penetrate the tissue to carry out suturing operation. This operation is required for each stitch. This puncture action greatly increases the risk of surgery because it is difficult to accurately determine the depth of penetration within the body. When this procedure is a gastric procedure, this spin-drilling and hooking device will likely cause damage to the outer tissue of the cavity, which greatly increases the patient’s surgical risk.

Moreover, the suturing device provided by the Apollo Endosurgery Inc. cannot realize the knotting operation of the suture without the aid of other devices after the suturing is completed. Thus, when the suture line is knotted, the suturing device must be matched with a specialized wire knotter. In practical operation, the device originally occupying one of the insertion tubes is removed, and then the wire knotter is inserted into the insertion tube by a special device to be fed into the body, after which the suture line is cut and the wire knotter is directly left in the body of the surgical patient. The knotting operation is not only troublesome, but also leaves a non-human rigid object inside the human body, which is easy to cause post-operative risks.

In addition, wound closure, tissue suturing and fixation of the digestive tract stent under an existing soft endoscope have the following problems: some instruments have large sizes and unsuitable, which are extremely easy to damage the mucosa of the digestive tract. For some instruments, one action needs to be broken down into multiple steps, multiple components need to be manually used, multiple exchanges of auxiliary instruments result in frequent motions, errors may occur, some parts need to be removed from the endoscope to be replaced. After that, the endoscope needs re-entry, which may double the operation and damage, and prolong the procedure. The sizes of the retaining clips cannot be adjusted (only few sizes). Too many clamping teeth means too much tissue damage, the fixing clip that does not pass through the insertion tube will block the field of view of the endoscope. Tearing of the trauma edge occurs during clamping, especially with fibrotic tissue or scarring tissue which are hard to close . When making the closing, it will happen that the alignment is inaccurate or the closing is too tight. The extent of the suture (large or small trauma) or suture depth (full or layered) is extremely difficult to adapt universally or be adjusted. If the position of clamping is too shallow, the clip is easily fallen off, while if the position of clamping is too deep, it will easily lead to tissue perforation. When applying force to the abdomen, injury by hard objects in the abdomen also needs to be prevented. The flexible endoscopy has no support (unlike the rigid laparoscope with support) and is difficult to knot directly. It’s hard to control the tension of the line. After the operation, granulation tissue hyperplasia, incomplete closure, leakage, bleeding and necrosis again lead to the second operation and even laparotomy. There are large number of components, the installation is time-consuming, and the cost is too much expensive .

### SUMMARY

In order to overcome at least one of the deficiencies of the prior art, there is provided a suture device, a treatment device having a suture device, and a treatment system.

In one aspect, the present invention provides a suture device comprising a main shell, a suture needle, a needle feeding assembly, and a needle anti-backoff assembly. One end of the suture needle is connected to a suture line, and the other end is a tip. The needle feeding assembly is disposed on the main shell and forms a separable engagement relationship with the suture needle to control advancement of the suture needle. The needle anti-backoff assembly is disposed on the main shell and forms a separable engagement relationship with the suture needle to prevent retraction of the suture needle.

According to an embodiment of the present invention, the needle feeding assembly comprises two pull cords, one pull cord controlling the advancement of the needle feeding assembly to drive the suture needle to advance, and the other pull cord controlling the retraction of the needle feeding assembly.

According to an embodiment of the invention, the needle feeding assembly comprises a pull cord, an upper shell, a spring, a needle feeding tooth and a sliding block, wherein the pull cord penetrates through the sliding block and the upper shell, the needle feeding tooth is installed in the sliding block, one end of the spring abuts against a surface of the needle feeding tooth, the needle feeding tooth, the sliding block and the spring are installed in the sliding groove of the main shell, and the upper shell covers the sliding groove.

According to an embodiment of the present invention, the needle anti-backoff assembly comprises a needle anti-backoff tooth, a lower shell and an elastic piece, wherein the needle anti-backoff tooth is mounted at the lower shell, the needle anti-backoff tooth and the lower shell are installed in the sliding groove of the main shell, and the lower shell and the elastic piece are fixed to the main shell.

In accordance with an embodiment of the present invention, the main shell includes a first housing having a connecting slot and a second housing having a connecting projection that is inserted into the connecting slot to connect the first housing and the second housing together.

In another aspect, the invention also provides a treatment device comprising an operating handle and a suture device. The suture device is secured to a distal end of an endoscope, the suture device comprises a main shell, a suture needle, a needle feeding assembly, and a needle anti-backoff assembly. One end of the suture needle is connected with a suture line, and the other end is a tip. The needle feeding assembly is disposed on the main shell and forms a separable engagement relationship with the suture needle to control advancement of the suture needle. The needle anti-backoff assembly is disposed on the main shell and forms a separable engagement relationship with the suture needle to prevent retraction of the suture needle. The operating handle is secured to an operating end of the endoscope, and the operating handle controls the needle feeding assembly to control the advancement of the suture needle.

In accordance with an embodiment of the present invention, an operating handle includes a safety switch assembly, an inner ring assembly, a clutch assembly, an operating lever assembly, and a torsion limiter assembly.

According to an embodiment of the present invention, the treatment device further comprises a connecting ring configured to be sleeved on the endoscope.

In accordance with an embodiment of the present invention, the treatment device further includes a snare ring configured to perform a cutting action onto tissue at the distal end of the endoscope.

In accordance with an embodiment of the present invention, the treatment device further includes a hook clamp configured to perform a clamping action onto tissue or a hooking action of the suture line at the distal end of the endoscope.

In another aspect, the invention also provides a treatment system comprising an endoscope and a treatment device. The treatment device is used in conjunction with the endoscope including an operating handle and a suture device. The suture device is secured to a distal end of the endoscope, the suture device comprises a main shell, a suture needle, a needle feeding assembly, and a needle anti-backoff assembly. One end of the suture needle is connected with a suture line, and the other end is a tip. The needle feeding assembly is disposed on the main shell and forms a separable engagement relationship with the suture needle to control advancement of the suture needle. The needle anti-backoff assembly is disposed on the main shell and forms a separable engagement relationship with the suture needle to prevent retraction of the suture needle. The operating handle is secured to an operating end of the endoscope, and the operating handle controls the needle feeding assembly to control the advancement of the suture needle.

According to an embodiment of the present invention, the treatment device further comprises a connecting ring configured to be sleeved on the endoscope.

In accordance with an embodiment of the present invention, the treatment device further includes a snare ring configured to perform a cutting action onto tissue at the distal end of the endoscope.

In accordance with an embodiment of the present invention, the treatment device further includes a hook clamp configured to perform a clamping action onto tissue or a hooking action of the suture line at the distal end of the endoscope.

In another aspect, the invention further provides a method for using a suture device, comprising the following steps:
operating a needle feeding assembly such that a suture needle and the needle feeding assembly move forward together, at that time a tip of the suture needle is exposed from a main shell, and the suture needle returns to the main shell after traveling a distance;
operating the needle feeding assembly such that the needle feeding assembly is moved rearward until being engaged with the suture needle, at that time an engaged position is close to the tip of the suture needle, and the suture needle is not movable under the action of a needle anti-backoff assembly;
operating the needle feeding assembly such that the suture needle and the needle feeding assembly move forward together until the tip of the suture needle is ready to be exposed from the main shell;
operating the needle feeding assembly such that the needle anti-backoff assembly is moved backwards until being engaged with the suture needle, at that time the engaged position is close to one end of the suture needle provided with a suture line, and the suture needle is not movable under the action of the needle anti-backoff assembly; and
repeating the above four steps to perform suturing of target suture tissue (such as wound tissue, etc.) or knotting of the suture line.

According to an embodiment of the present invention, the needle feeding assembly comprises a first pull cord and a second pull cord, wherein the four steps correspond to pulling the first pull cord, pulling the second pull cord, pulling the first pull cord and pulling the second pull cord, respectively.

According to an embodiment of the invention, operating the needle feeding assembly is completed by operating an operating handle.

In accordance with an embodiment of the present invention, prior to operating the needle feeding assembly, the tissue is snared with a snare ring at a distal end of an endoscope.

In accordance with an embodiment of the present invention, after all of the steps described above are completed, a knot tying action of the suture line is accomplished by clamping two ends of the suture line using a hook clamp at the distal end of the endoscope.

In view of the foregoing, the suture device, treatment device and treatment system of the present invention can be used for natural orifice transluminal surgery (non-invasive surgery), minimally invasive surgery, or invasive surgery. The suture device is simple in structure and low in manufacturing cost, the advancing of the suture needle can be controlled only through the needle feeding assembly, intermittent suturing or continuous suturing is carried out in soft tissue, the operation is simple, and the risk of surgical failure is greatly reduced. In addition, the suture device of the present invention also includes a needle anti-backoff assembly enabling continuous advancement of the suture needle. In particular, the needle feeding assembly and the needle anti-backoff assembly of the present invention are in a separable engagement relationship with the suture needle, so that complex functions (e.g., suturing of the target suture tissue and knotting of the suture line) can be achieved through a simple structure, the occupied volume of the suture device is small, and the manufacturing cost is low. In the prior art, if the suturing of the tissue in the body is required to be in-vitro operation, the suturing function can be achieved only by matching a spin-drilling and hooking device in the whole process, and the existing spin-drilling and hooking device spins and hooks the tissue directly and then pull it up. The suture device in the present invention does not need to adopt the spin-drilling and hooking device in the suturing process, so that the suturing function can be achieved independently, the damage to the tissue by the spin-drilling and hooking device is avoided, and the surgical risk is greatly reduced.

In addition, in one embodiment, the needle feeding assembly may include two pull cords. The two pull cords can be respectively controlled to control movement of the needle feeding assembly in different directions, the operation is simple and clear, and the risk of surgical failure is greatly reduced. In one embodiment, the treatment device and treatment system of the present invention may also include a snare ring and a hook clamp, which enable various functions such as cutting, suturing, knotting, and the like of the tissue together with the suture device. In one embodiment, the treatment device and treatment system of the present invention may further include a connecting ring that can secure the external insertion tube and the endoscope together when the external insertion tube is used, and the connecting rings can be distributed at multiple points along the hose of the endoscope, thereby dispersing the stress. The existing treatment device of Apollo Endosurgery Inc have only a distal end and an operating end fixed, and is extremely easy to cause passive bending of the endoscope.

In particular, the suture device, treatment device and treatment system provided by the present invention can achieve knotting in a manner close to the knotting habits operated in the open surgery after the target suture tissue is stapled. It has been difficult to have a knot tying operation in the body since the force applied in the body is only upward, which will pull the target suture tissue. If the force is too large, the tissue is damaged, while if the force is too small, the knot is not tight, and if the tissue moves, it is likely that the knot is loose and the suture needs to be re-sutured. The suture device, treatment device, and treatment system provided by the present invention achieve knotting with a simple operation similar to that of suturing. The suture line is guided by the suture needle to form a “knot”, and finally the two ends of the suture line are tensioned by the hook clamp, and the applied force during the knotting is horizontal or nearly horizontal. In this way, the tightness of the knot is moderate, the wound faces are aligned accurately, the operation is simple, the operation difficulty of surgery is reduced, no clamp will be left in the human body, and the difficulty of knotting in the prior art is overcome. The device in the present invention can be used to close and suture different areas of wounds or tissues, either in layers or in full layers, as needed.

In order to make the above and other objects, features and advantages of the invention more obvious and easier to understand, the following is a preferred embodiment. The detailed description is as follows in conjunction with the attached drawings.

### BRIEF DESCRIPTION OF THE DRAWINGS

FIG. 1A is a schematic diagram of a suture device according to an embodiment of the present invention;

FIG. 1B is another perspective view of the suture device provided in accordance with an embodiment of the present invention;

FIG. 2A is a schematic diagram of a main shell of the suture device according to an embodiment of the present invention;

FIG. 2B is a schematic view of a first housing and a second housing after separation shown in FIG. 2A;

FIG. 2C is another perspective view of the main shell of the suture device according to an embodiment of the present invention;

FIG. 3A is a schematic view of a needle feeding assembly of the suture device provided in accordance with an embodiment of the present invention (wherein only a portion of the first and second pull cords are shown);

FIG. 3B illustrates an exploded view of the needle feeding assembly of the suture device according to an embodiment of the present invention;

FIG. 3C is an assembled state diagram of the needle feeding assembly and the main shell of the suture device provided in accordance with an embodiment of the present invention;

FIG. 4A is a schematic view of a suture needle of the suture device provided in accordance with an embodiment of the present invention;

FIG. 4B is another perspective view of the suture needle of the suture device provided in accordance with an embodiment of the present invention;

FIG. 4C is a side view of the suture needle of the suture device provided in accordance with an embodiment of the present invention;

FIG. 5A illustrates an exploded view of a needle anti-backoff assembly of the suture device according to an embodiment of the present invention;

FIG. 5B is a partially assembled schematic view of the needle anti-backoff assembly of the suture device provided in accordance with an embodiment of the present invention;

FIG. 5C is an assembled state diagram of the needle anti-backoff assembly, the suture needle and the main shell of the suture device provided in accordance with an embodiment of the present invention;

FIGS. 6A-6D are schematic diagrams illustrating different states of the needle anti-backoff assembly, the suture needle and the needle feeding assembly of the suture device during the movement process provided in accordance with an embodiment of the present invention;

FIG. 7A is a schematic diagram of an operating handle provided in accordance with an embodiment of the present inventio;

FIG. 7B is another perspective view of the operating handle provided in accordance with an embodiment of the present invention;

FIG. 8A is an exploded schematic view of a safety switch assembly of the operating handle provided in accordance with an embodiment of the present invention;

FIG. 8B is an installation schematic view of the safety switch assembly of the operating handle provided in accordance with an embodiment of the present invention;

FIG. 9A illustrates an exploded view of an inner ring assembly of the operating handle provided in accordance with an embodiment of the present invention;

FIG. 9B is an installation schematic view of the inner ring assembly of the operating handle provided in accordance with an embodiment of the present invention;

FIG. 10A is an exploded view of a clutch assembly of the operating handle provided in accordance with an embodiment of the present invention;

FIGS. 10B-10F are schematic diagrams illustrating the installation of the clutch assembly and a front shell and a rear shell of the operating handle provided in accordance with an embodiment of the present invention;

FIG. 11A is an exploded view of an operating lever assembly of the operating handle provided in accordance with an embodiment of the present invention;

FIGS. 11B-11D are schematic diagrams illustrating the installation of the operating lever assembly and the front shell and the rear shell of the operating handle provided in accordance with an embodiment of the present invention;

FIG. 12A is an exploded schematic view of a torsion limiter assembly of the operating handle provided in accordance with an embodiment of the present invention;

FIG. 12B and FIG. 12C are schematic diagrams illustrating the installation of the torsion limiter assembly and a front shell and a rear shell of the operating handle provided in accordance with an embodiment of the present invention;

FIG. 13A is a schematic diagram illustrating the installation of the operating handle and an endoscope according to an embodiment of the present invention;

FIG. 13B is another perspective view showing the installation of the operating handle and the endoscope provided in accordance with an embodiment of the present invention;

FIGS. 14A-14G are assembled schematic diagrams of an operating handle according to another embodiment of the present invention;

FIG. 15A is a schematic diagram showing the installation of a connection ring and the endoscope according to an embodiment of the present invention;

FIG. 15B is a schematic diagram showing the installation of the connection ring, the suture device and the endoscope according to an embodiment of the present invention;

FIGS. 16A and 16B are schematic diagrams illustrating the installation of the operating handle and the endoscope in accordance with an embodiment of the present invention;

FIGS. 17A and 17B illustrate an installation process of the suture device and the endoscope according to an embodiment of the present invention;

FIGS. 18A and 18B, 19A-19C, 20A and 20B, 21A-21C, 22A-22C, and 23A-23M are schematic diagrams illustrating use of a treatment system provided in accordance with an embodiment of the present invention;

FIG. 24 is a schematic diagram of a snare ring of the treatment device provided in accordance with an embodiment of the present invention;

FIG. 25 is a schematic diagram of a suture device according to another embodiment of the present invention; and

FIG. 26 is an exploded view of a suture device according to another embodiment of the present invention.

### DETAILED DESCRIPTION OF THE INVENTION

The present invention provides a suture device, an operating handle cooperating with the suture device, a treatment device consisting of the suture device and the operating handle, a treatment system consisting of the treatment device and an endoscope cooperating therewith. The suture device can be used to suture in vivo on the tissue of a mammal, whether the object is a person and whether the subject is alive. The suture device provided by the invention not only can be used in cooperation with an endoscope, but also can be matched with other devices capable of achieving steering guidance.

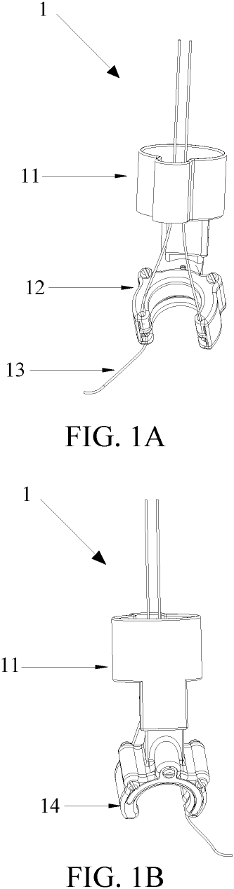

As shown in FIGS. 1A and 1B, the present invention provides a suture device 1 for piercing and stapling tissue to perform a surgical procedure within a body. The suture device 1 includes a main shell 11, a needle feeding assembly 12, a suture needle 13, and a needle anti-backoff assembly 14.

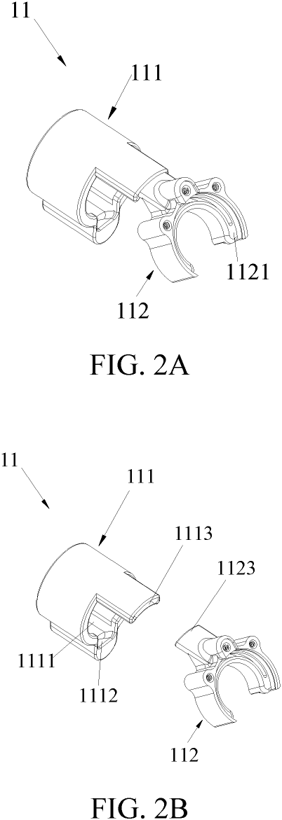

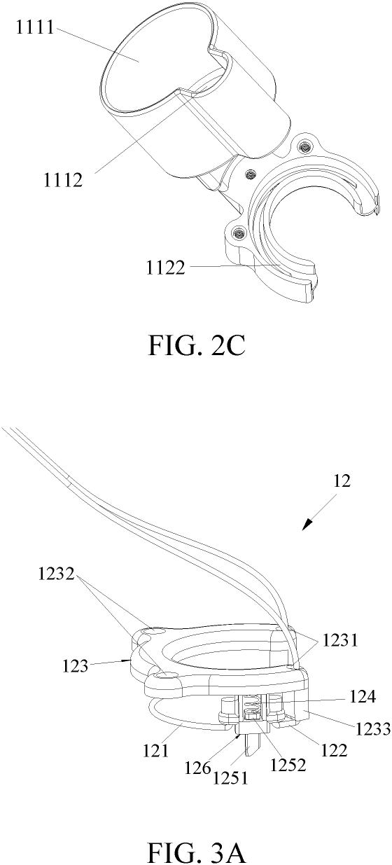

In the present embodiment, as shown in FIGS. 2A-2C, the main shell 11 includes a first housing 111 and a second housing 112. The middle of the first housing 111 may have two channels 1111, 1112 when the suture device of the present embodiment is used in conjunction with an endoscope with a single insertion tube, the first channel 1111 may be sleeved on the distal end of the endoscope, and the second channel 1112 may allow an external insertion tube to pass through thus to secure the external insertion tube. However, the present invention is not limited thereto. When the suture device is used in conjunction with an endoscope with dual insertion tubes, the first housing may have only one channel so that the suture device can be sleeved on the distal end of the endoscope. In another embodiment, the first housing may not have a channel but may be secured to the distal end of the endoscope by other structures, such as a fixing band.

The shape of the first housing 111 and the second housing 112 is not limited in the present invention. Preferably, the outer surfaces of the first housing 111 and the second housing 112 of the present embodiment are smooth surfaces and do not have edges and corners. By this arrangement, the suture device does not affect the field of view of the distal end of the endoscope as far as possible, it is convenient for suturing operation at the distal end in the body, and damage to the tissue in the body is also reduced. In addition, the outer surface is arcuate, and it is also avoided that the edges of the housing cause damage to the tissue in vivo during operation.

In the present embodiment, the first housing 111 has a connecting groove 1113, the second housing 112 has a connecting protrusion 1123, and the shape and size of the connecting protrusion 1123 are matched with the shape and size of the connecting groove 1113. The connecting protrusion 1123 is inserted into the connecting groove 1113 to connect the second housing 112 and the first housing 111 together. In the present embodiment, the connecting groove 1113 is curved in shape, so that the main shell 11 of the suture device can be close to the distal end of the endoscope as much as possible without a convex structure to affect the field of view of the distal end of the endoscope. However, the present invention is not limited thereto. In other embodiments, the connection slots may be other shapes as long as the connection relationship between the first housing 111 and the second housing 112 is achieved. In other embodiments, the second housing has a connecting groove, and the first housing has a connecting protrusion.

In the present embodiment, in order to firmly connect the first housing 111 and the second housing 112, a laser welding mode may be used to weld the connecting groove 1113 and the connecting seams formed after the connecting protrusion 1123 are joined together. The present invention is not limited to the fixed manner of the first housing 111 and the second housing 112. In another embodiment, the main shell may not be divided into two portions of the first housing 111 and the second housing 112, and the main shell may be formed in an integrally formed manner. The main shell 56 is divided into two parts, so that each component is simpler to manufacture, easy to standardize, and lower in production cost.

In the present embodiment, the second housing 112 has a first sliding groove 1121 and a second sliding groove 1122, and the first sliding groove 1121 and the second sliding groove 1122 are respectively located on two sides of the main body part of the second housing 112.

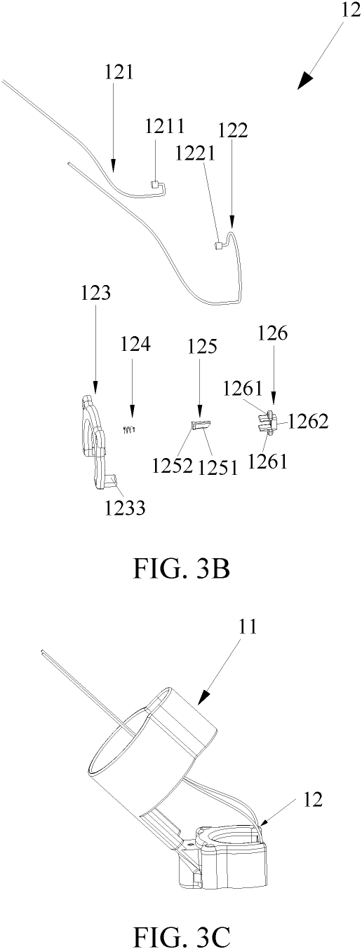

In the present embodiment, as shown in FIGS. 3A-3C, the needle feeding assembly 12 is disposed on the main shell 11 and forms a separable engagement relationship with the suture needle 13 to control advancement of the suture needle 13, thereby performing a suture operation or a knot tying operation of the target suture tissue, such as wound tissue, etc. In the present embodiment, the needle feeding assembly 12 includes a first pull cord 121, a second pull cord 122, an upper shell 123, a spring 124, a needle feed tooth 125, and a sliding block 126.

In the present embodiment, the needle feeding assembly 12 includes two pull cords, the first pull cord 121 and the second pull cord 122. The two pull cords are respectively controlled to control movement of the needle feeding assembly in different directions, the operation is simple and easy to understand, and the risk of surgical failure is greatly reduced. However, the present invention is not limited thereto. In other embodiments, the needle feeding assembly 12 may have only one pull cord, and the advancement of the suture needle can be controlled by retracting and pulling the one pull cord. In that case, others structures of the needle feeding assembly may be changed correspondingly, for example, one pull cord may enable bi-directional control by gear sliding or linkage rotation. In another embodiment, the needle feeding assembly 12 may not employ a pull cord to enable control of the suture needle by alternative means of other structures.

In the present embodiment, one end of the first pull cord 121 and the second pull cord 122 each have a stop block 1211, 1221, which can be a cord knot or a specially made plastic block in the practical application. The present invention does not limit the form and material of the stopper, so long as the diameter of the stop is greater than the diameter of the cord portion of the first and second pull cords 121, 122. In other embodiments, the first pull cord 121 and the second pull cord 122 may not have a stop, and one end of the first pull cord 121 and the second pull cord 122 may be secured to the sliding block 126 in an adhesive manner.

In the present embodiment, the upper shell 123 has two through holes 1231 and two upper fixing holes 1232. The through holes 1231 allow the cord body portion of the first pull cord 121 and the second pull cord 122 to pass through, and the upper fixing holes 1232 allow fixing member (such as screws) to pass through thus to fix the upper shell 123 to the second housing 112 of the main shell 11. Preferably, the upper shell 123 also has two extending portions 1233 when the upper shell 123 is covered on the second sliding groove 1122 of the second housing 112, the extending portions 1233 just wraps the side surface, thereby reducing the possibility of damage to the tissue in the body after the components and the components are assembled. However, the specific shape of the upper shell 123 is not limited in this disclosure.

In the present embodiment, the needle feeding tooth 125 includes a tooth portion 1251 and a stopper 1252, the top end of the tooth portion 1251 is an inclined surface, and may be clamped on a slot of the suture needle 13 (specifically set forth below). One end of the spring 124 abuts against the stopper 1252 and the other end abuts against the upper shell 123.

In the present embodiment, the sliding block 126 has two side holes 1261 and one intermediate hole 1262. The two side holes 1261 can pass through the cord body portion of the first pull cord 121 and the second pull cord 122, but clamp the stop blocks 1211, 1221 of the first pull cord 121 and the second pull cord 122, i.e., the outer diameter of the stop blocks 1211, 1221 is larger than the inner diameter of the side holes 1261. The intermediate hole 1262 can be passed through the teeth 1251 of the needle teeth 125, but the stopper 1252 of the needle teeth 125 cannot pass through the intermediate hole 1262.

In actual use, the first pull cord 121 and the second pull cord 122 pass through the side holes 1261 of the sliding block 126, and then penetrate through the through holes 1231 of the upper shell 123 and are mounted in the middle hole 1262 of the sliding block 126, and one end of the spring 124 abuts against the surface of the needle feeding tooth 125. The needle entry teeth 125, the sliding block 126, and the spring 124 are mounted together within the second sliding groove 1122 of the second housing 112 and can slide within the second sliding groove 1122, with the upper shell 123 covering the second sliding groove 1122. One end of the first pull cord 121 and the second pull cord 122 is fixed to the sliding block 126, the other end of the first pull cord 121 and the second pull cord 122 are fixed to the operating handle, and the needle feeding teeth 125 penetrate into the sliding block 126. When pulling the pulling cord, one end of the tooth portion of the needle feeding tooth 125 clamps the clamping groove on the suture needle, and then drives the suture needle to move forwards, so that the needle enters the needle, the needle pulling, the threading, and the loop reciprocating.

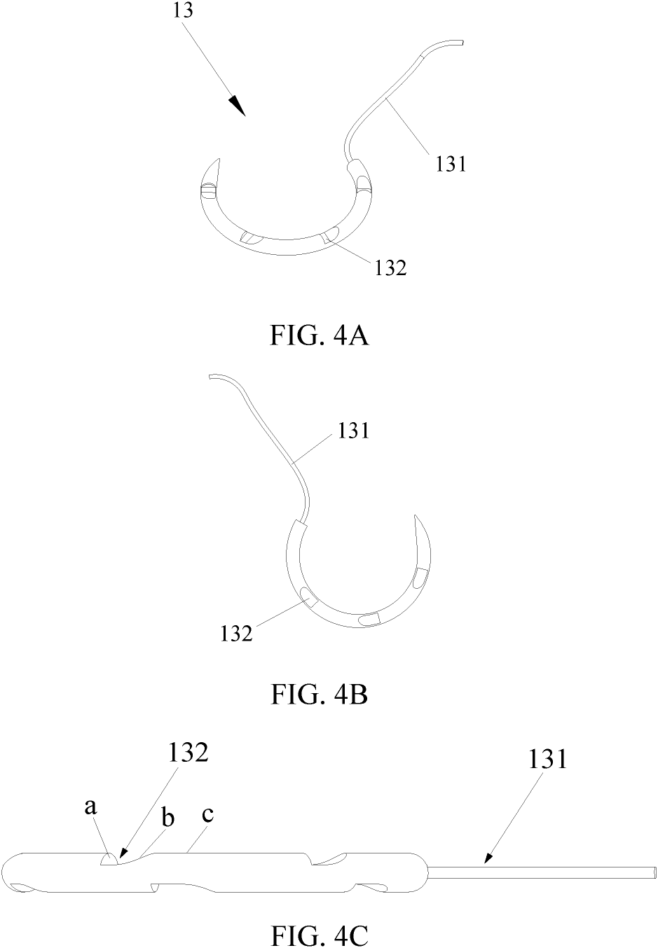

In the present embodiment, as shown in FIGS. 4A-4C, the suture line 131 may be secured or removably secured to one end of the suture needle 13, and the other end of the suture needle 13 is a tip that pierces the tissue. In the present embodiment, one side of the suture needle 13 has four clamping grooves 132, and the other side of the suture needle 13 is provided with three clamping grooves 132 Through the arrangement, the needle feeding teeth 60 degrees and 120 degrees can be fed, so that the advancing stroke control of the suture needle 13 is more flexible. If only the needle is required to control the needle 180°, there is no need to set such a plurality of slots. In the present embodiment, in order to prevent the thickness of the suture needle from becoming thin, the clamping grooves on both sides are arranged in a staggered manner, i.e., the clamping grooves are not completely correspondingly arranged on the two sides of the same place. With such an arrangement, the suture needle is not easily bent or broken. However, the present invention is not limited thereto.

The teeth 1251 of the needle teeth 125 are detachably engaged in the slots 132 of the suture needle 13. The present invention is not limited in number to the number of slots 132. In the present embodiment, each of the clamping grooves 132 is an asymmetric structure, one side is an inclined surface A with a larger inclination angle, a blocking effect is achieved, and the other side is an inclined surface B with a small inclination angle, and the inclined surface B and the inclined surface of the top end of the tooth portion 1251 are matched. The top end of the tooth portion 1251 is obliquely arranged, and the tooth portion 1251 slides out of the clamping groove 132 through the inclined surface B under the action force of the pull cord, so as to release the engagement relationship between the needle feeding tooth 125 and the suture needle 13. In this embodiment, the included angle between the slope A of each card slot 132 and one side C of the suture needle 13 is 60°-90°, and the included angle between the other side ramp B and the side C of the suture needle 13 is 10°-45°. However, the degree of inclination of the present invention is not limited in any way. In the present embodiment, the inclined surface B is not a flat surface, but is provided with an arc-shaped surface, so that the top end of the tooth portion 1251 slides out of the clamping groove 132 so as to release the engagement relationship between the needle feeding tooth 125 and the clamping groove 132.

The suture needle 13 of the present embodiment is a wire suture needle. Specifically, the tail of the suture needle 13 has a conical depression, one end of the suture line 131 is inserted into the conical recess, and then the tail end of the suture needle 13 is squeezed, so that the suture line 131 is fixed at the tail end of the suture needle 13. However, the present invention is not limited thereto. In other embodiments, the suture may be removable secured to one end of the suture needle in other ways, such as a conventional puncture needle. In the present embodiment, the suture needle 13 can be made of stainless steel, and the diameter of the cross section of the suture needle 13 can be 8 mm, 10 mm, 12 mm, etc. However, the invention is not limited thereto, and can be selected according to actual treatment requirements. In the present embodiment, the length of the suture line 131 is 30 cm to 60 cm, the suture line 131 can be made of polypropylene, nylon, etc. and the length and the material of the suture are not limited. In other embodiments, bio absorbable suture can be used, so that suture removal is not necessary and the risk of secondary surgery can be reduced.

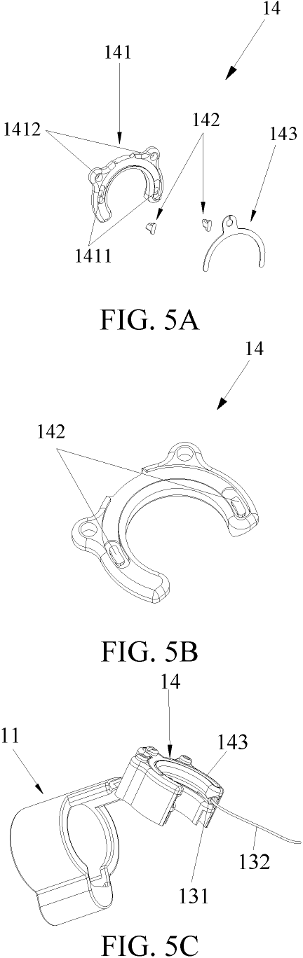

In the present embodiment, as shown in FIGS. 5A-5C, the needle anti-backoff assembly 14 is disposed on the main shell 11 and forms A separable engagement relationship with the suture needle 13 to prevent retraction of the suture needle 13. The needle feeding assembly 12 and the needle anti-backoff assembly 14 are located on both sides of the suture needle 13, respectively.

In the present embodiment, the needle anti-backoff assembly 14 includes a lower shell 141, a needle anti-backoff tooth 142, and an elastic piece 143, the lower shell 141 is provided with two positioning holes 1411 and two lower fixing holes 1412, the two needle anti-backoff teeth 142 are respectively installed in the two positioning holes 1411, and the lower fixing hole 1412 is used for fixing the lower shell 141 to the second housing 112 of the main shell 11. The elastic sheet 143 is also provided with a fixing hole for fixing the elastic sheet 143 to the second housing 112 of the main shell 11In actual assembly, the suture needle 13 is first disposed in the first sliding groove 1121 of the second housing 112, then the lower shell 141 with the needle anti-backoff tooth 142 is covered on the first sliding groove 1121, and then the elastic piece 143 and the second housing 112 are fixed.

The top end of the needle anti-backoff teeth 142 may be snapped onto the slot 132 of the suture needle 13. In the present embodiment, the top ends of the two needle anti-backoff teeth 142 are beveled. The slope is matched with the inclination angle of the inclined surface B of the clamping groove 132, so that the needle anti-backoff teeth 142 can slide out of the clamping groove 132 through the inclined surface B under the acting force of the pull cord, and the needle feeding teeth 125 and the needle anti-backoff teeth 142 are respectively located on the two sides of the suture needle 13 and separately clamped on the clamping groove 132 of the suture needle 13. The separable engagement of the fingers herein refers to that the engagement relationship can be formed as well. Both the needle feeding tooth 125 and the needle anti-backoff tooth 142 and the suture needle are separable snap-fit relationships, so complex functions can be achieved by a simple structure (e.g., knotting of the suture and suture of the target suture tissue), the size of the suture device is small, and the manufacturing cost is low. The present invention does not limit the shape of the tip of the needle teeth 125 and the needle anti-backoff teeth 142. In other embodiments, the top ends of the needle teeth 125 and the needle anti-backoff teeth 142 may also be arc-shaped to facilitate sliding. It is also within the scope of the present invention to be able to achieve the function of the present invention as long as the snap-fit relationship can be formed with the suture needle and the snap-fit relationship can be released.

In the present embodiment, elastic force is applied to the needle anti-backoff teeth 142 by virtue of the elastic force of the elastic pieces 143, so that the needle anti-backoff teeth 142 can slide out of the clamping grooves 132 under the action of external force, and the needle anti-backoff teeth 142 can be pressed by the elastic force to clamp the needle anti-backoff teeth 142 in the clamping grooves 132.

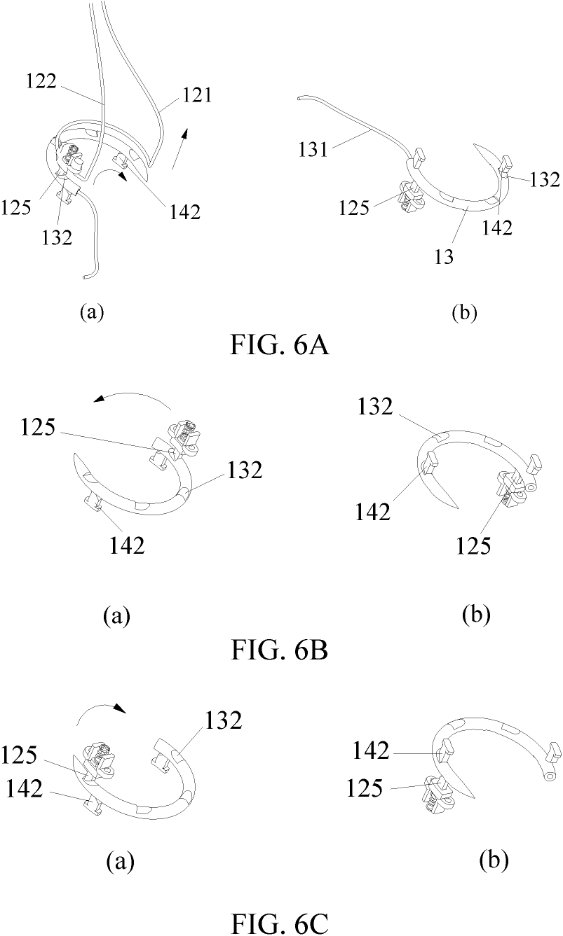

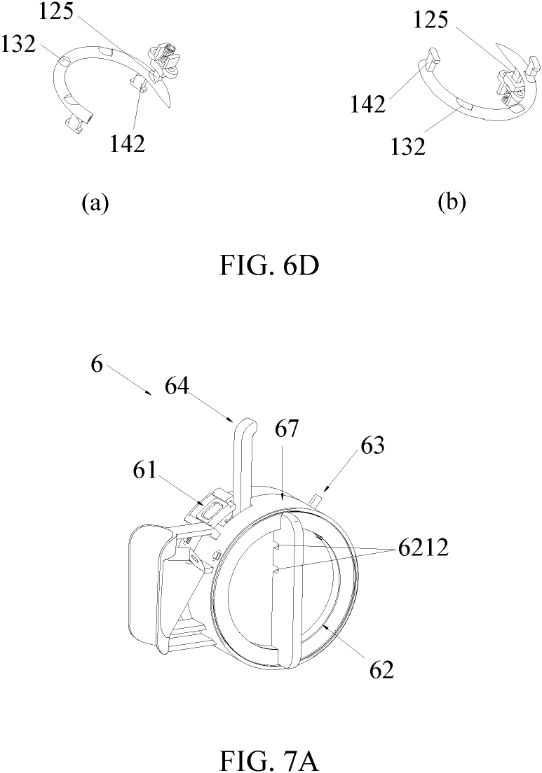

To more clearly illustrate how the suture device provided by the embodiments of the present invention performs the suturing action of the target suture tissue, the following is described in conjunction with FIGS. 6A-6D. The left (a) and right (b) of each figure of FIGS. 6A-6D are top view and bottom view relationships. In order to clearly illustrate the position of the internal parts, all of the elements are not shown in the drawings. The “back side” referred to in the following description is from the perspective of the left (a) in FIGS. 6A-6D, for example, as defined above in (a) as a positive face, below the back, since (a) and (b) are mirror-like relationships, the back side is shown above in (b).

As shown in FIG. 6A, in the initial state, the needle teeth 125 of the needle feeder assembly 12 are engaged in one of the slots 132 of the suture needle 13 in the initial state, and the engagement position is at the tail end of the suture needle 13, i.e., close to one end of the suture line. One of the needle anti-backoff teeth 142 of the needle anti-backoff assembly 14, as shown in FIG. 6A (b), is clamped within one of the slots 132 on the back of the suture needle 13, which is located at the top end of the suture needle 13, i.e., near one end of the suture needle tip. At this time, the first pull cord 121 is pulled, since the needle teeth 125 snap into the slot 132 of the suture needle 13, and the direction of the force is such that the top end of the needle feeding tooth 125 abuts against one side of the slot 132. Under the action of the external force, the pull cord 121 pulls the needle feeding teeth 125 so as to drive the entire suture needle 13 forward in a clockwise direction as shown in FIG. 6A (a). The needle anti-backoff tooth 142 is counter-clockwise relative to the running direction of the suture needle 13 (actually the needle anti-backoff tooth 142 is not moving, but the suture needle 13 moves clockwise), and the needle anti-backoff tooth 142 can slide out of the clamping groove through the inclined surface of the clamping groove 132 without any obstruction to the travel of the suture needle 13. In this process, the tip of the suture needle 13 is exposed from the main shell 11, then the suture needs to be stapled to drive the suture to pass through the target suture tissue, after which the tip of the suture needle 13 is returned to the first sliding groove 1121 of the main shell 11.

At this time, the position of each part is shown in FIG. 6B, since the needle feeding teeth 125 have slid to the end of the second sliding groove 1122, the advancing cannot continue, and the first pull cord 121 cannot be pulled continuously. The force applied by the first pull cord 121 and the force applied by the second pull cord 122 are opposite in the opposite direction for the feed needle teeth 125. The user then pulls the second pull cord 122, which slides the ramp through the slot 132 out of the card slot 132 and then back in the counterclockwise direction as shown in FIG. 6B (a) until the most starting position is returned. In this process, the other needle anti-backoff tooth 142 (which is located on the left side as shown in FIG. 6B) is clamped in a slot 132 on the back of the suture needle 13. Since the top end of the needle anti-backoff tooth 142 abuts against one side of the angle of inclination of the clamping groove 132, the needle anti-backoff tooth 142 does not disengage from the clamping groove 132, and the suture needle 13 does not have a reverse motion.

As shown in FIG. 6C, the needle feeding tooth 125 return to the initial position, the second pull cord 122 will not continue to pull the needle feeding tooth 125, and the needle feeding tooth 125 again snap into one of the slots 132 of the suture needle 13, which is located near one end of the suture needle tip. Correspondingly, the needle anti-backoff tooth 142 on the left side shown in FIG. 6C (b) continue to be snapped into the card slot 132 on the back of the suture needle 13, i.e., the snap position is located near one end of the suture needle tip. Next, the first pull cord 121 is pulled, because the direction of the force is such that the top end of the needle feeding tooth 125 abuts against one side of the slot 132 under the action of the external force, the pull cord 121 pulls the needle feeding tooth 125, thereby driving the entire suture needle 13 to continue in a clockwise direction. In this process, the just-engaged needle anti-backoff tooth 142 slide out of the card slot 132 through the bevel of the card slot 132 without any obstruction to the travel of the suture needle 13.

When the needle feeding tooth 125 has slid to the end of the second sliding groove 1122, the advancement cannot be continued, and the first pull cord 121 cannot continue to pull, at which point the suture needle is thus able to complete a turn of travel as shown in FIG. 6D. At this point, the engagement position of the needle feed tooth 125 and the suture needle 13 is located near one end of the suture needle tip, and the needle anti-backoff tooth 142 on the right side shown in FIG. 6D is clamped within the slot 132 of the suture needle 13.

The second pull cord 122 is then pulled to repeat the above-described action and the suture needle may be brought together with the suture to complete the threading of the suture or suture (threading needle) of the target suture tissue. As can be seen in FIGS. 6A-6D, the needle feeding tooth 125 are alternately clamped at the slots of the top or trailing end of the suture needle, with the needle feeding tooth 125 and the suture needles 13 moving together, also with relative movement (the needle 13 is not moving, moving the needle feeding tooth 125). The needle anti-backoff tooth 142 does not move, and the suture needle 13 is moved relative to the needle anti-backoff tooth 142 such that the two needle anti-backoff teeth 142 alternate with one of the slots 132 of the suture needle 13.

The present invention also provides a method for using a suture device, comprising the following steps:
operating the needle feeding assembly such that the suture needle and the needle feeding assembly move forward together, at which point the tip of the suture needle is exposed from the main shell and the suture needle returns to the main shell after traveling a distance;
operating the needle feeding assembly such that the needle feeding assembly is moved rearward until being engaged with the suture needle, at which point the engaged position is close to the tip of the suture needle, and the suture needle is not actuated under the action of the needle anti-backoff assembly;
operating the needle feeding assembly such that the suture needle and the needle feeding assembly move forward together until the tip of the suture needle is ready to be exposed from the main shell;
operating the needle feeding assembly so that the needle anti-backoff assembly is moved backwards until being engaged with the suture needle, at the moment, the engaged position is close to one end of the suture needle provided with a suture line, and the suture needle is not movable under the action of the needle anti-backoff assembly; and
repeating the above four steps to perform suturing of target suture tissue (such as wound tissue, etc.) or knotting of the suture line.

In particular, the method of using the suture device provided by the present invention comprises the following steps.

In the initial state, the needle teeth of the needle feeding assembly engage the slots of the suture needle. The first pull cord of the needle feeding assembly is operated such that the suture needle and the needle feeding assembly move forward along the sliding groove. In this process, the needle feeding assembly moves within the main shell from one end of the sliding groove to the other end, with the tip of the suture being exposed from the main shell, traveling a segment of travel (e.g., half-circle) back into the main shell. The term “forward” in “move forward” in this application is not a straight line direction. When “forward” refers to the movement in a clockwise direction, “backward” refers to the movement in a counter-clockwise direction. When “forward” refers to the movement in a counter-clockwise direction, “backward” refers to movement in a clockwise direction.

At this point, the needle feeding assembly cannot continue to slide, and the first pull cord cannot continue to be pulled. The second pull cord of the needle feeding assembly is then pulled such that the needle feeding assembly is moved rearward, and the needle feeding assembly moves within the main shell from just the other end of the sliding groove to the beginning of the beginning until no removement. In this process, the needle anti-backoff assembly is clamped to the push suture needle, the engaged position is close to the tip of the push suture needle, and the push suture needle is not actuated under the action of the needle anti-backoff assembly in this process.

The first pull cord of the needle feeding assembly is then operated such that the suture needle and the needle anti-backoff assembly travel forward, during which the needle feeding assembly moves again from one end of the sliding groove to the other end within the main shell, at which point the tip of the suture needle is ready to be exposed from the main shell;

Continuing to operate the second pull cord of the needle feeding assembly such that the needle feeding assembly is moved rearward until it is engaged with the suture needle and cannot move, at which point the engaged position is close to one end of the suture needle with the suture. In this process, the needle anti-backoff assembly is clamped to the suture needle, the engaged position is close to the tip of the suture needle, and the suture needle is not actuated under the action of the needle anti-backoff assembly in this process.

In one embodiment, the act of operating the needle feeding assembly is accomplished by operating the operating handle.

In one embodiment, prior to operating the needle feeding assembly, the tissue is looped with a snare ring at the distal end of the endoscope.

In one embodiment, after all of the above steps are completed, it is also included to clamp both ends of the suture to complete the knotting action of the suture using a hook clamp at the distal end of the endoscope.

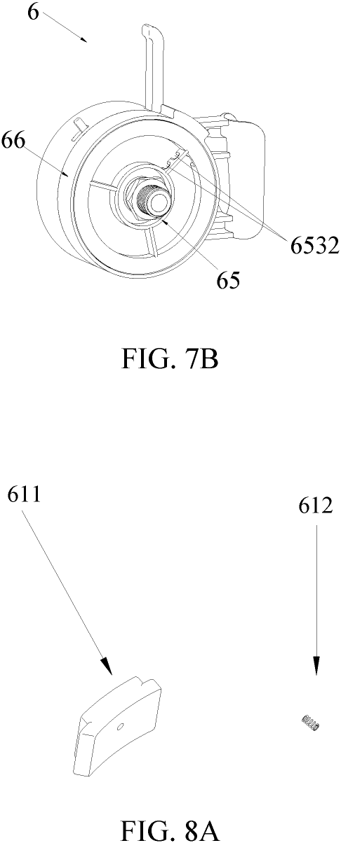

In another aspect, the invention also provides a treatment device comprising the above-mentioned suture device 1 and an operating handle 6 as shown in FIGS. 7A and 7B. The handle 6 provided by the embodiment is used with the suture device 1 provided in this embodiment. In the present embodiment, the operating handle 6 includes a safety switch assembly 61, an inner ring assembly 62, a clutch assembly 63, an operating lever assembly 64, a torsion limiter assembly 65, a rear shell 66, and a front shell 67.

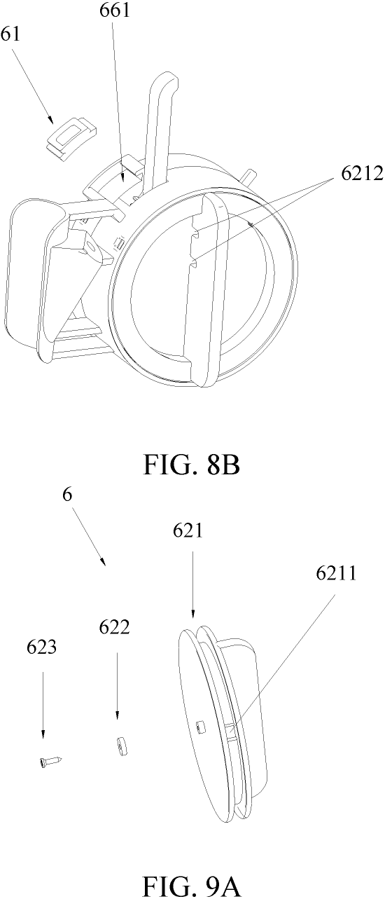

As shown in FIGS. 8A and 8B, the safety switch assembly 61 includes a safety switch 611 and a compression spring 612 that loads the compression spring 612 into the aperture of the safety switch 611 and is then mounted to the safety switch slot 661 of the rear shell 66.

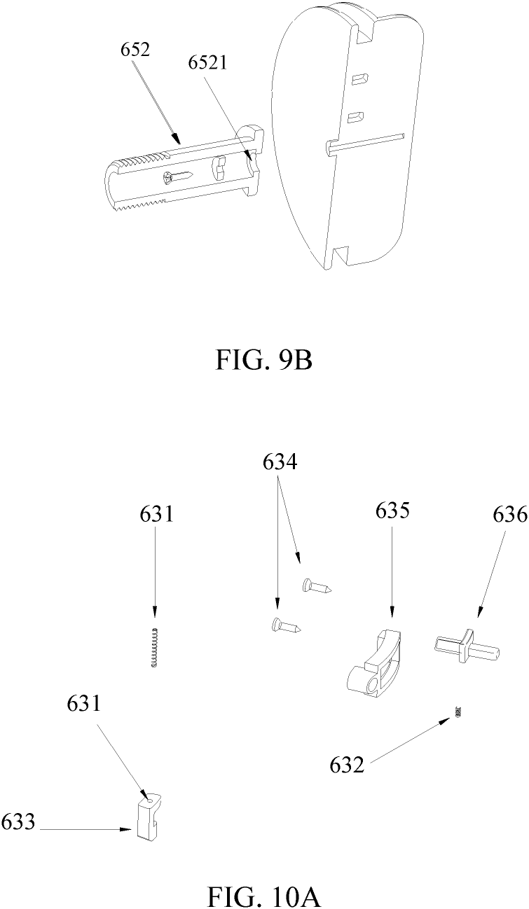

As shown in FIGS. 9A and 9B, the inner ring assembly 62 includes an inner ring 621, a washer 622, and a screw 623 inserting the inner ring 621 into the central bore 6521 of the central shaft 652, mounting the washer 622, and tightening the screw 623. In this embodiment, the inner ring 621 has a square hole 6211 and two tie holes 6212 (see FIG. 7A).

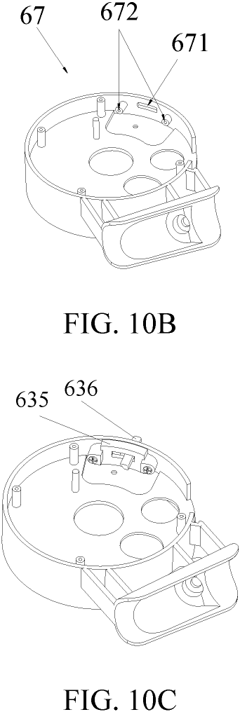

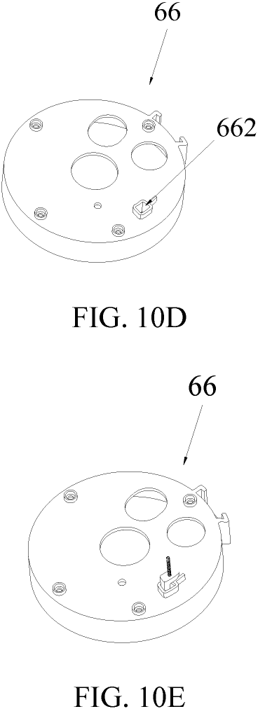

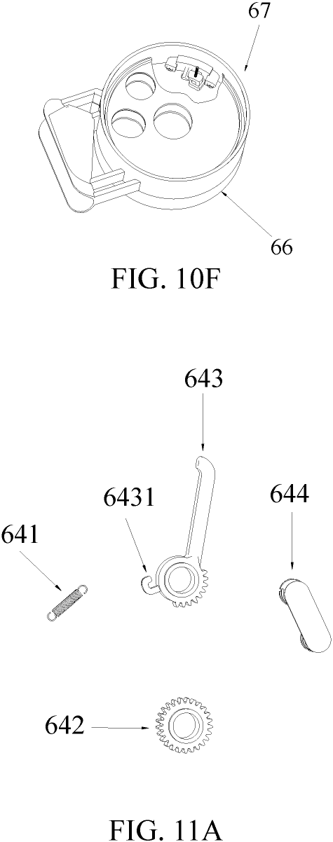

As shown in FIGS. 10A-10F, the clutch assembly 63 includes compression springs 631 and 632, a limiting pin 633, a screw 634, a limiting member 635, an operating handle clutch lever 636, which fits the compression spring 632 into the bore of the operating handle clutch lever 636, together into the limiting member 635, together into the side hole 671 of the front shell 67, and screws 634 into the screw hole 672. The compression spring 631 is loaded into the limit pin 633, together into the square hole 662 of the back shell 66.

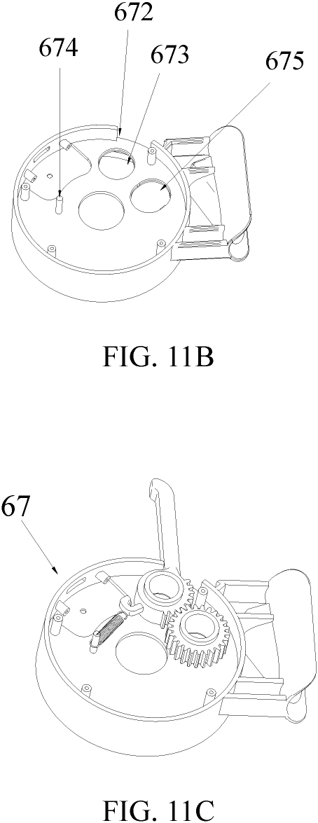

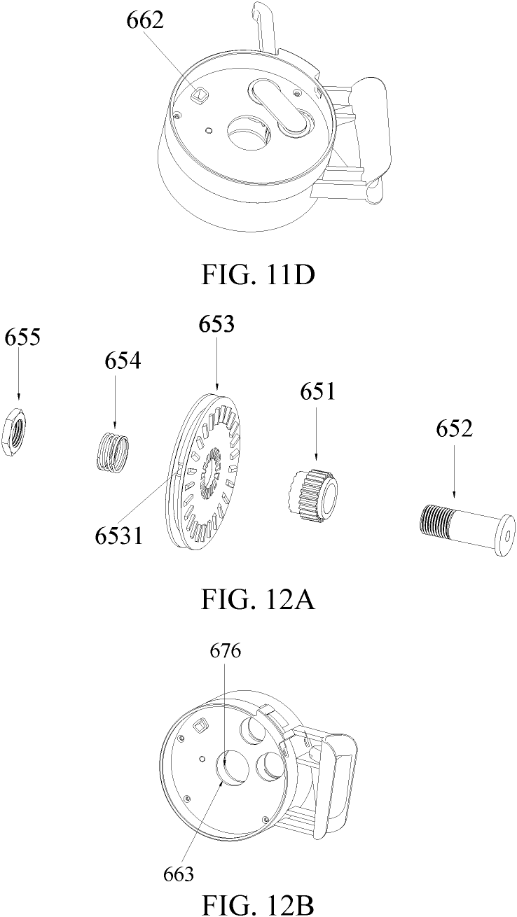

As shown in FIGS. 11A-11D, the lever assembly 64 includes a tension spring 641, a transmission gear 642, an operating handle pull rod 643, and a gear connection block 644 mounted to the hook 6431 of the operating handle pull rod 643, together into the side slot 672 and the bottom circular hole 673 of the front shell 67, and the tension spring 641 is mounted in the cylinder 674 of the front shell 67, and the transmission gear 642 is mounted in the bottom elliptical hole 675 of the front shell 67. The compression spring 631 is loaded into the limit pin 633, together into the square hole 662 of the back shell 66. After the upper rear shell 66 is covered, the gear connection block 644 is loaded into the operating handle pull rod 643, the transmission gear 642.

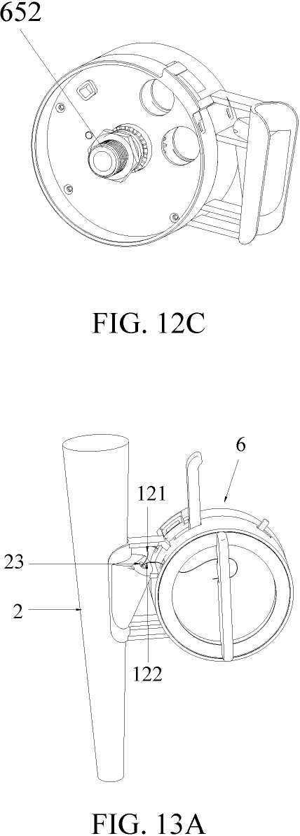

As shown in FIGS. 12A-12C, the torsion limiter assembly 65 includes a grooved gear 651, a central shaft 652, an outer ring 653, a compression spring 654, a hex nut 655 fitting the central shaft 652 into the central circular hole 676 of the front shell 67, a central circular hole 663 of the rear shell 66 aligned with the central shaft 652, and an outer ring 653, a compression spring 654, and a hex nut 655 aligned with the central shaft 652. In this embodiment, the outer ring 653 has a square hole 6531 and two tie holes 6532 (see FIG. 7B).

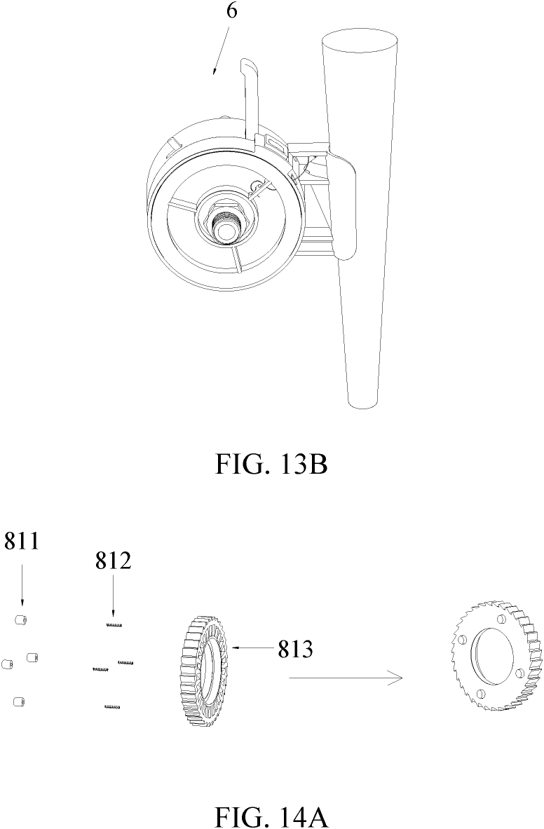

In another aspect, the invention also provides a treatment system comprising the above-mentioned treatment device and an endoscope 2 treatment device for use in conjunction with an endoscope, including an operating handle and a suture device. FIGS. 13A and 13B are schematic views of the operating handle 6, the first pull cord 121, and the second pull cord 122 mounted to the flexible endoscope 2.

The first pull cord 121 and the second pull cord 122 pass out of the insertion jaws 23 of the flexible endoscope 2, and are bonded to the inner and outer rings 621 and 653, respectively, through the holes of the handle 6. The handle 6 is mounted to the insertion jaw 23 position of the flexible endoscope 2 and is secured by a handle fastener.

The operating principle of the operating handle is to push the safety switch assembly 61 away, pull the operating handle pull rod 643 in the lever assembly 64, transfer the force to the transmission gear 642 through the gear connection block 644, and then to the grooved gear 651 in the torsion limiter assembly 65 to drive the outer ring 653 to rotate, thereby driving the first pull cord 121, wherein the tension spring 641 can reset the lever assembly 64 to achieve continuous pulling. The compression spring 654 can limit the transfer of excessive force to disengage the grooved gear 651 and the outer ring 653, thereby protecting the first pull cord 121 without breaking due to excessive force. The operating handle clutch lever 635 in the clutch assembly 63 is then slid to separate the stop pin 632 from the outer ring 653, to which the ring 653 may be free to rotate. The inner circular ring 621 in the inner circular ring assembly 62 drives the second pull cord 122.

The specific structure of the operating handle is not limited, so long as the function of operating the handle of the embodiment can be realized, and the handle can be matched with the suture device provided by the embodiment. In other embodiments, the suture device may also be used alone regardless of the operating handle. The suture device may have a pull ring disposed at the end of the pull cord, pulling the pull cord by pulling the pull ring.

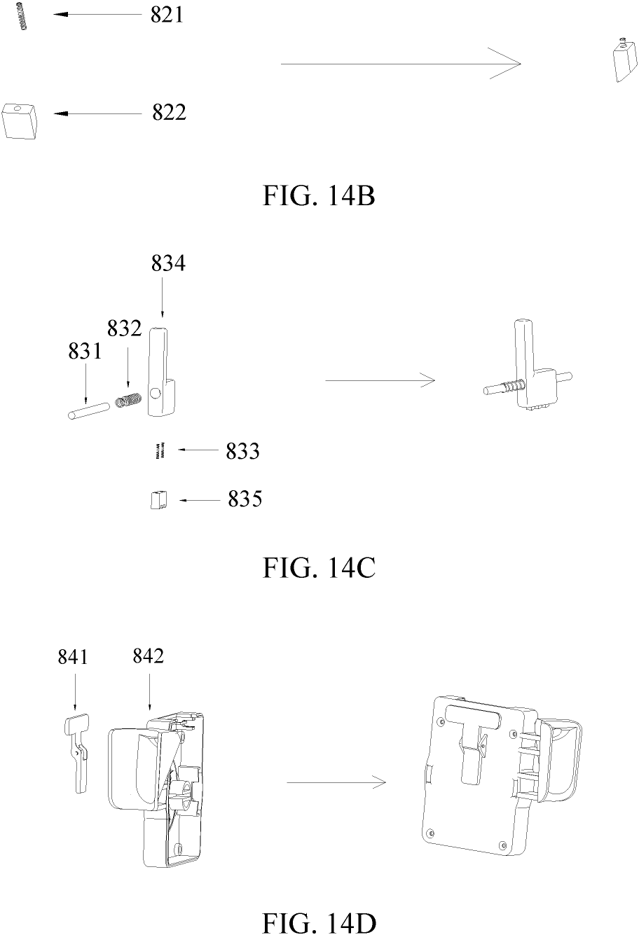

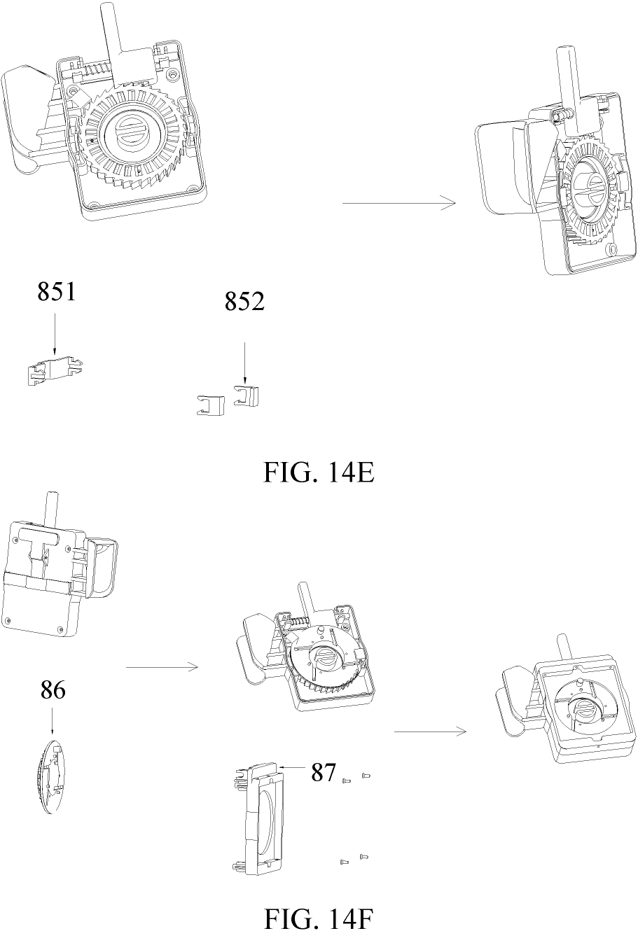

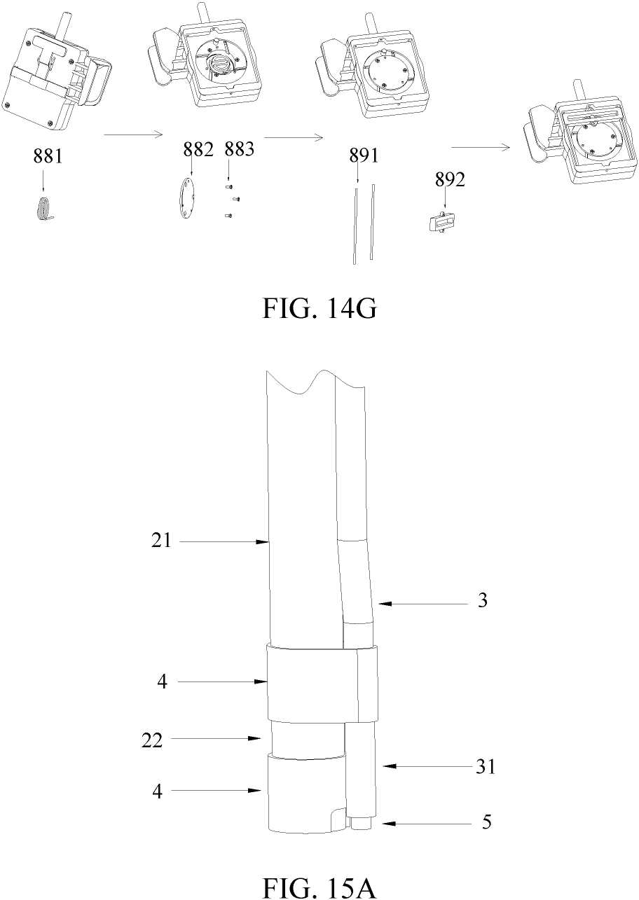

As shown in FIGS. 14A-14G, another embodiment of the present invention provides an operating handle 8 operating handle comprising a spring cap 811, a screw 812, a drive wheel 813, a spring 821, an anti-backoff tooth 822, a shaft 831, a spring 832, 833, a pull rod 834, a screw 835, a key 841, a rear shell 842, a first connector 851, a second connector 852, a driven wheel 86, a front shell 87, a torsion spring 881, a cover plate 882, a screw 883, a shaft 891, and a sliding block 892.

Pulling the pull rod 834 drives the drive wheel 813 to rotate counterclockwise. The pull rod 834 is released, the spring 832 resets the pull rod 834 to achieve a reciprocating pull, and the drive wheel 813 continues to rotate counterclockwise. The anti-backoff teeth 822 are configured to prevent the drive wheel 813 from rotating clockwise when the pull rod 834 is released. By pressing the key 841, the driving wheel is driven to move along the axis by the first connector 8511 and the second connector 852 (as shown on the left side in the figure). The drive wheel 813 rotates in a counterclockwise direction to drive the driven wheel 86 to rotate together. The driving wheel 813 is driven to move along the axis (as shown on the left side in the figure) by the first connecting member 8511 and the second connecting member 852, at which time the driving wheel 813 and the driven wheel 86 are separated. The drive wheel 813 rotates in a counterclockwise direction and will drive the driven wheel 86 to rotate together. By pressing the key 841, the driving wheel is driven to move along the axis by the first connecting member 8511 and the second connecting member 852 (as shown on the left side in the figure), at which point the driving wheel 813 and the driven wheel 86 are separated. The torsion spring 881 drives the driven wheel 86 to rotate clockwise. The installation of operating handle is completed. Rotation of the driven wheel 86 drives the sliding block 892 to slide along the shaft 891. There by driving the tensioning and relaxation of the two pull cords of the suture device.

In contrast to the operating handle of the previous embodiment, the operating handle provided by the present embodiment requires only a slight force during operation, and one-handed operation can be achieved, and only two fingers of a single hand can be completed, and the action direction is changed from multi-direction to one-way, the size is reduced, the manufacturing link is reduced, and the cost is reduced.

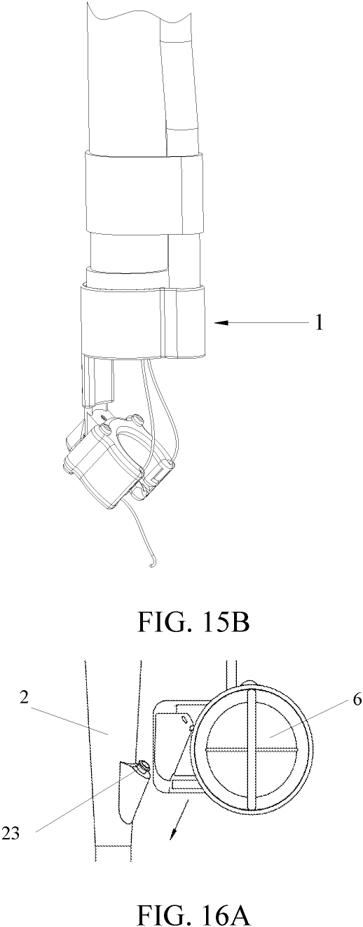

In the present embodiment, the treatment device may further comprise a connecting ring 4, which is made of silica gel, with an elastic force. However, the present invention does not limit the material of the connecting ring. As shown in FIGS. 15A and 15B, the external insertion tube 3, the push tube 5, the connecting ring 4, and the suture device 1 are mounted to the endoscope 2. Specifically, the external clamp tube 3 is fixed to the hose portion 21 of the endoscope 2 by the connecting ring 4, and the push tube 5 is mounted in the inner hole of the external insertion tube 3. Another connecting ring 4 is sleeved on the distal end 22 of the endoscope 2 and the suture device 1 sleeves the connecting ring 4 to secure the suture device 1 to the distal end 22 of the endoscope 2 and the head end 31 of the external insertion tube 3 is inserted into the suture device 1. The connecting ring 4 in this embodiment can function as both a fixed external clamp tube 3 and an endoscope 2, and can increase the connection strength of the first end 22 of the endoscope 2, so that the suture device 1 can be firmly fixed at the front end 22 of the endoscope 2 in actual use, and the plurality of connecting rings can be distributed in a multi-point manner along the hose of the endoscope so as to disperse the stress. An existing treatment device of a surgical company in the Apollo et al. is only fixed at the distal end and the operating end and is extremely easy to assist in assisting in the passive bending of the endoscope.

In the present embodiment, the treatment device may also include a snare ring 7, as shown in FIG. 24. The snare ring 7 cuts tissue at the distal end of the endoscope, or cuts the tissue by current. The target suture tissue is then stapled using the suture device provided in this embodiment. The snare ring 7 may be inserted through its own insertion tube, or may be incorporated by an endoscope external insertion tube, which is not intended to be any limitation.

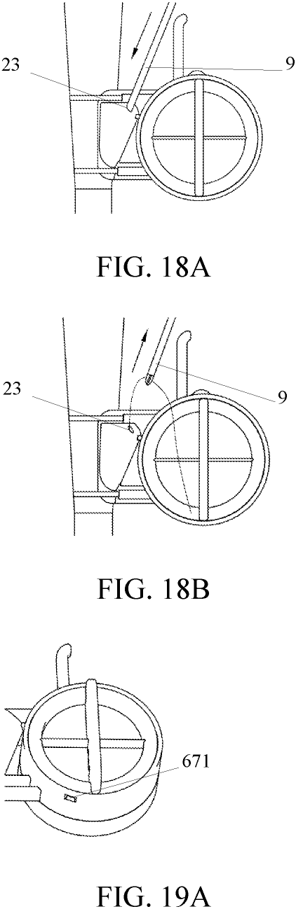

In the present embodiment, the treatment device may further include a hook clamp 9 (as shown in FIG. 18B), the hook clamp 9 performing a clamping action or a hooking action of the suture at the distal end of the endoscope, and finally clamping both ends of the suture to complete the knotting action of the suture. The forceps may be inserted through the jaws of the endoscope itself, or may be provided by an endoscope external jaw, which is not limited in this respect. In practical use, the two jaws can be simultaneously extended into two hooks for operation, and only one hook clamp can be used for operation. In the present embodiment, the front end of the hook is a hook with a hook.

The treatment device and the treatment system provided by the embodiment can realize various functions such as cutting, suturing, knotting and the like of the tissue through the snare ring, the suture device and the hook forceps. In addition, the treatment device and the treatment system provided in the present embodiment may further comprise a biological adhesive, a plugging adhesive, a stent, a staple and an anastomosis clip, thereby realizing the diversification of functions.

How to install the treatment device of the present invention onto an endoscope is described next. Alternatively, the endoscope may be used in conjunction with an endoscope having an end diameter of 98 mm, a jaw aperture of less than or equal to 2 mm, and a working length of ≦ 1300 mm. The present invention is not limited to the model of the endoscope.

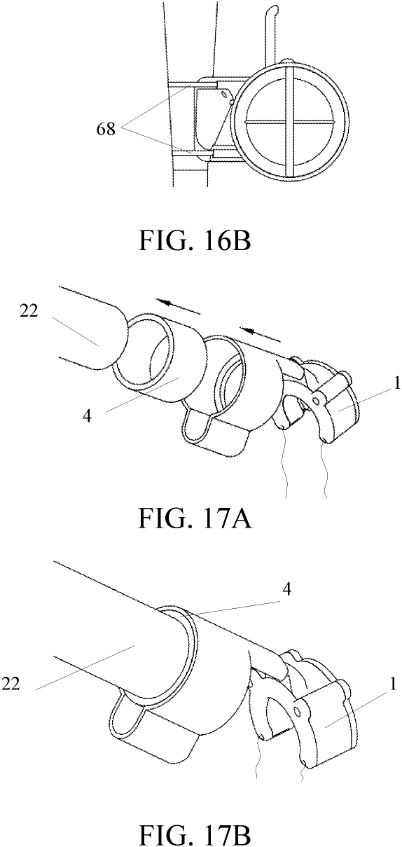

First, as shown in FIGS. 16A and 16B, the operating handle 6 is mounted to the operating end of the endoscope 2 and the operating handle 6 and the endoscope 2 are securely fastened together by a handle fastener 68. In the present embodiment, the handle fastener 68 is in the shape of a belt by bypassing the endoscope 2 to fasten the operating handle 6 and the endoscope 2. However, the present invention does not limit the shape of the handle fastener 68 as long as the components capable of securing the function can be used as handle fasteners.

Next, as shown in FIGS. 17A and 17B, the connecting ring 4 is sleeved on the distal end 22 of the endoscope 2, and then the suture device 1 is sleeved on the connecting ring 4 located at the front end 22 of the endoscope 2. The endoscope 2 is opened and the direction of the suture device 1 is adjusted to a C-shaped opening downward under the monitor. After the adjustment position, the suture device 1 is tightened until the loop step surface of the suture device 1 abuts the distal end 22 of the endoscope 2.

As shown in FIGS. 18A and 18B, the hook clamp 9 is inserted from the jaws of the endoscope 2, through the jaws to the distal end of the endoscope 2, then the first pull cord 121 of the suture device is clamped, and the first pull cord 121 at the proximal end is pulled into the jaws. The hook clamp 9 is then removed and pulled out from the insertion jaw 23 to bring one end of the first pull cord 121 out of the insertion jaw 23 (the arrow pointing to the direction of movement of the hook clamp 9). The two pull cords are partitioned at the wire outlet above the suture device 1. Preferably, the ends of the first and second pull cords may be labeled with different colors. The present invention is not limited in any way, and in other embodiments, the first and second pull cords may be identical to the operator, and the operator is in the area. In further embodiments, the first pull cord and the second pull cord may be identified by material, color, etc. It is convenient for the zones to be identified.

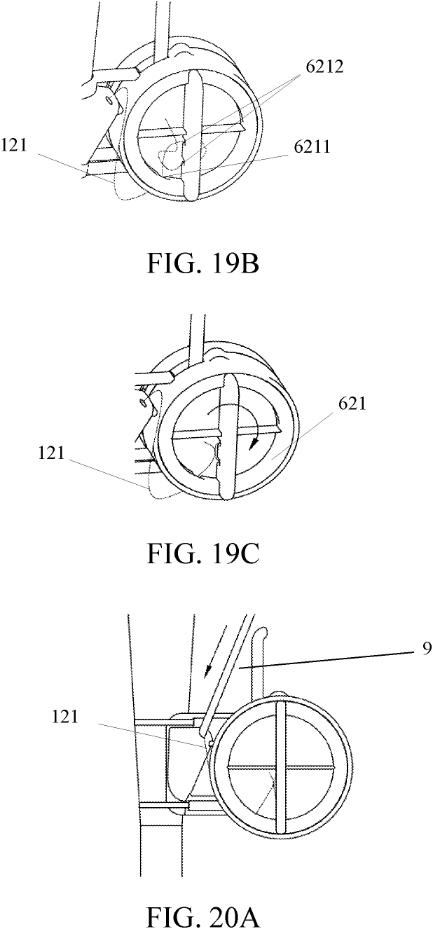

As shown in FIGS. 19A-19C, the square hole 6211 of the inner circular ring 621 of the operating handle 6 is aligned with the side hole 671 of the front shell, the first pull cord 121 that has been pulled out of the insertion jaw 23 penetrates into the square hole 6211 of the operating handle, and then the two tie holes 6212 on the wheel wing of the inner circular ring 621 are tied). The inner circular ring rotates clockwise, loose the first pull cord 121 out of the wire outside the handle, and is wound into the circular groove of the inner circular ring 621.

As shown in FIGS. 20A and 20B, the hook clamp 9 is again inserted from the insertion jaw 23 of the endoscope 2, through the jaws to the distal end of the endoscope 2, then the second pull cord 122 of the suture device is clamped, and the second pull cord 122 located near the distal end is pulled into the jaws. The hook clamp 9 is then removed and pulled out from the jaws to bring one end of the second pull cord 122 out of the jaw.

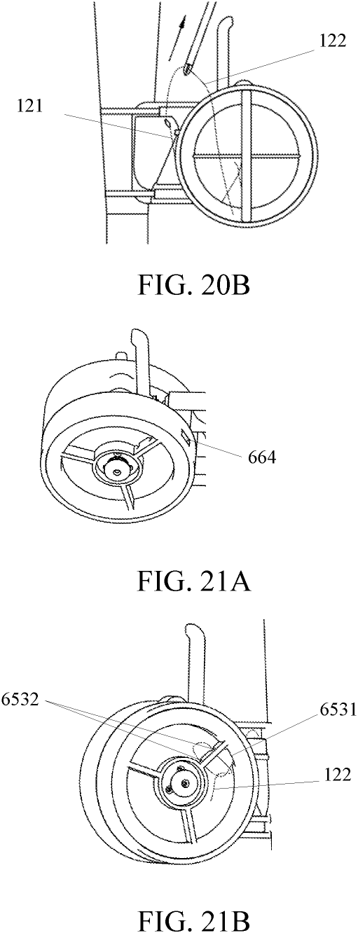

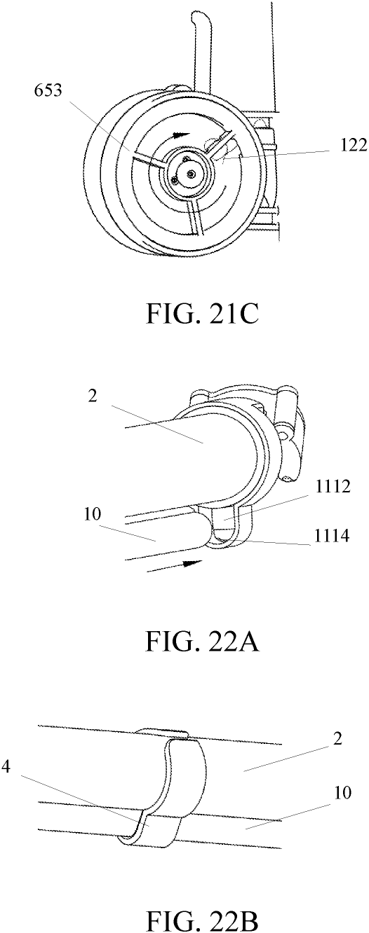

As shown in FIGS. 21A-21C, the square hole 6531 of the outer circular ring of the operating handle is aligned with the side hole 664 of the rear shell, the first pull cord 121 that has been pulled out of the insertion jaw is inserted into the square hole of the operating handle, and then the two tie holes 6532 on the wheel wing of the outer ring 653 are tied. The outer circular ring is rotated clockwise; the second pull cord 122 is loosely wound outside the handle, wound into the circular groove of the outer ring 653.

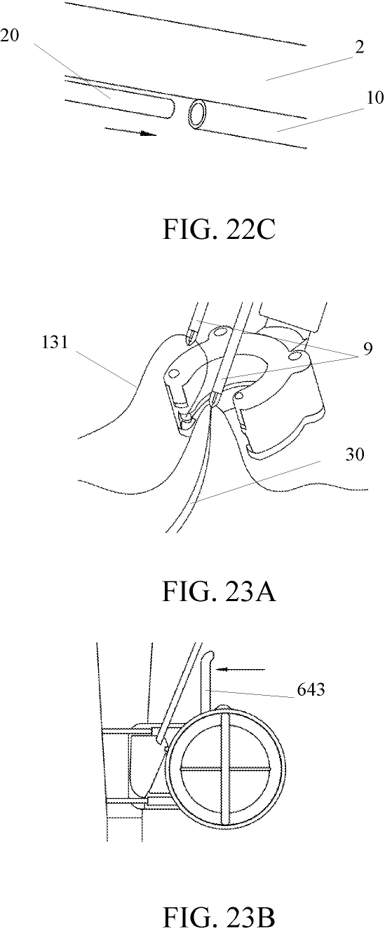

In an embodiment, endoscope 2 may be a single insertion tube endoscope. As shown in FIGS. 22A-22C, an external insertion tube 10 is first mounted to the endoscope 2. The front end of the external insertion tube 10 is inserted into the second channel 1112 of the first housing edge of the suture device until abutting against the stepped surface 1114 of the second channel 1112. A plurality of connecting rings is distributed along the jaw direction, and the external insertion tube 10 and the hose portion of the endoscope 2 are fastened by a connecting ring. The push tube 20 is inserted into the external insertion tube 10.

In another embodiment, the endoscope may be a dual insertion tube endoscope. At this point, it is not necessary to specifically mount the external insertion tube. Because the endoscope has two jaws, it is only necessary to insert the push tube into the empty jaws.

Next, explain how the suture device of the present invention performs suture.

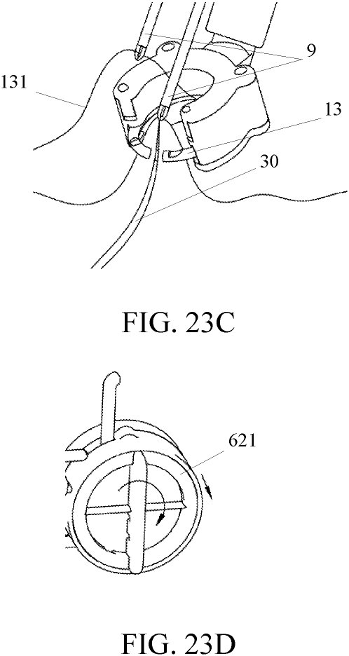

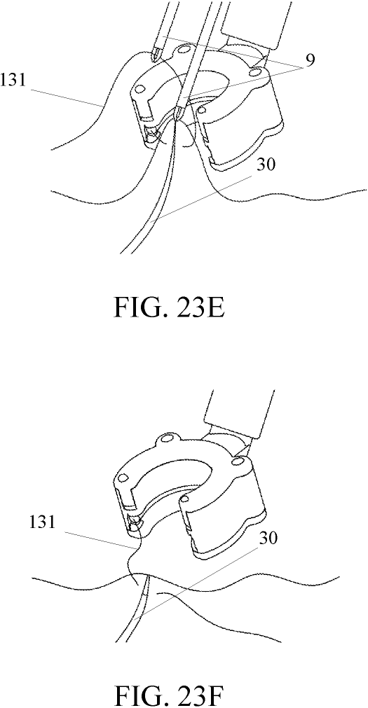

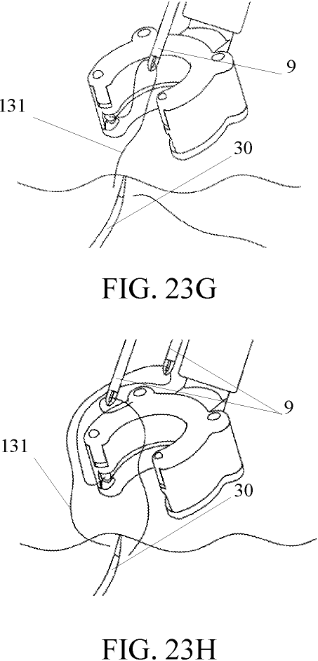

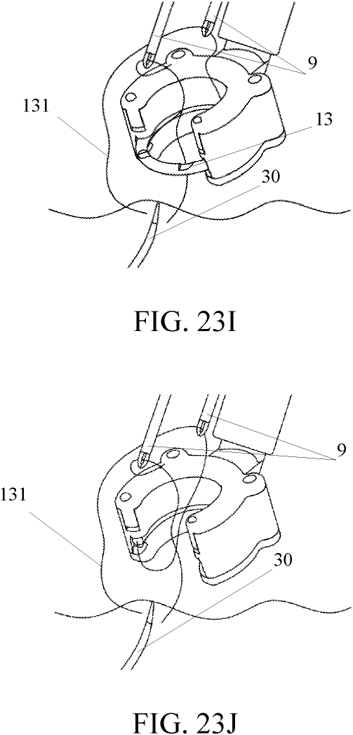

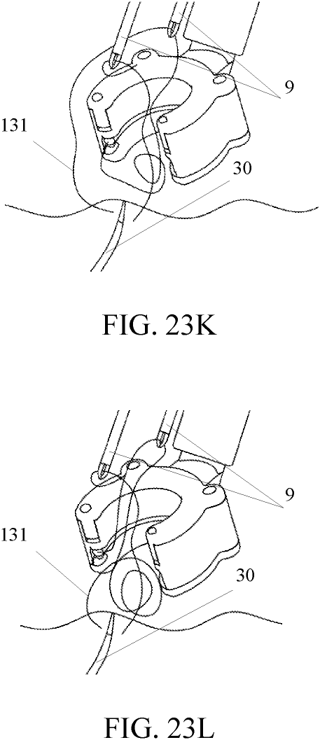

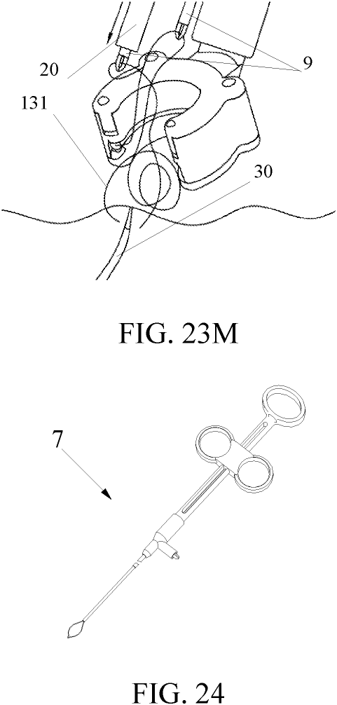

First, as shown in FIG. 23A, the hook clamp 9 is inserted from the insertion jaw of the endoscope, through the jaws to the distal end of the endoscope, and then the suture line 131 is found and clamped into the push tube to reach the distal end of the endoscope, the edge of the target suture tissue 30 is clamped, and the target suture tissue 30 is clamped in the middle of the suture device C-shaped opening (two pull cords in the figure are omitted). As shown in FIGS. 23B and 23C, the operating handle pull rod 643 is continuously pulled back to pass the suture needle through the tissue. The depth (full or layered) of the suture depends on the treatment of the wound or non-invasive tissue.

As shown in FIGS. 23D and 23E, after the suture needle 13 is rotated by 180° within the needle channel (i.e., the first sliding groove), the pull-back of the pull rod 643 is stopped and then the knob of the inner circular ring 621 of the operating handle is lifted by hand to pull out about 3 mm while rotating clockwise to tighten the second pull cord 122. The returnable handle pull rod 643 causes the suture needle 13 to rotate 180° within the first sliding groove and returned to its initial position by pulling back. The knob of the inner circular ring 621 of the handle is then lightly lifted to pull out about 3 mm while rotating clockwise to tighten the second pull cord 122. For a larger suture target, one side of the target tissue may be taken and sutured at first, then the other side of the target tissue is taken and sutured, and the suture line is properly tensioned and knotted. As shown in FIG. 23F, two hook clamps 9 are released. As shown in FIG. 23G, the hook clamp 9 in the insertion tube hooks the suture line 131 at the distal end (near the suture line) and moderately hooks and pulls the suture line 131 to pass through the tissue. As shown in FIG. 23H, the hook clamp 9 in the other push tube clamps the tail end of the suture line 131, adjusts the position of the suture device on the endoscope 2, so that the tail end of the suture line 131 is on the inner side of the suture C-shaped opening and the head end of the suture line 131 is on the outer side of the suture C-shaped opening.

As shown in FIG. 23I, the handle pull rod is continuously pulled back, while the pull-back resistance of the pull rod is increased after the suture needle is seen to be about the half circle through the monitor, i.e., the pull-back pull rod is stopped. The inner ring knob of the handle is operated with a hand to pull out while the clockwise rotation pulls the second tether. As shown in FIG. 23J, the handle pull rod is continuously pulled back, while the pull-back resistance of the pull rod is increased, i.e., the pull-back pull rod is stopped. The inner ring knob of the handle is operated with a hand to pull out while the clockwise rotation pulls the second tether. As shown in FIG. 23K, to this point the suture needle is rotated a circle, and the suture needle returns to the initial position. The steps described in this section are repeated, i.e., the suture needle is operated to re-rotate a circle. Repeating the suturing steps described above may be performed continuously.

As shown in FIG. 23L, the hook clamp 9 clamped to the end of the suture head is loosened, after the suture needle passes, the knot is quickly moved to the front of the suture head end, the suture line 131 is clamped again, as shown in FIG. 23M, the two hook clamps 9 are tightened, and the knot is gradually pushed by the knot pushing tube 20 to realize knotting. In order to ensure that the knot tying is firm enough, the knotting operation within this section can be repeated to complete the triple junction.

Exiting the push tube 20 and the two hook clamps 9, the endoscope is sheared, inserted into the endoscope empty jaw, and the suture line is cut off. After the suturing is complete, the endoscope and the auxiliary tool are withdrawn, and the suture device is removed.

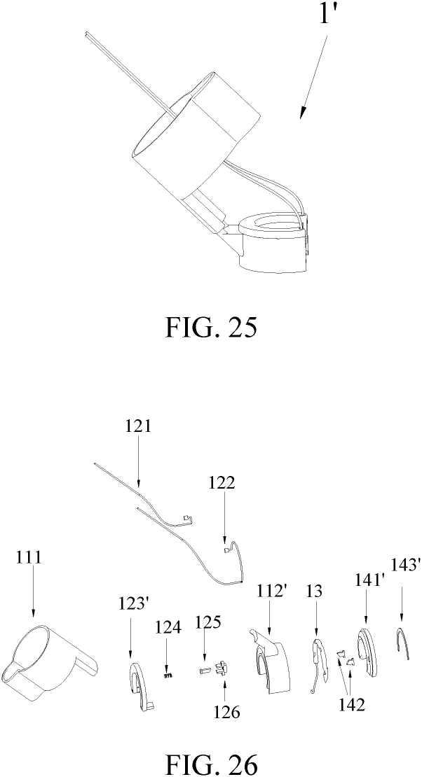

As shown in FIGS. 25 and 26, another embodiment of the present invention provides a suture device. The suture device 1′ provided by the present embodiment is substantially the same as the structure of the suture device 1 provided in the previous embodiment. Elements that are the same as in the previous embodiment are numbered the same, but are described below only. Each element of the suture device 1′ of the present embodiment is in laser welding connection, so that the arrangement of screws and lugs is omitted, the number of parts of the device is saved, the production cost is reduced, and the assembly time is saved. In particular, the upper shell 123′ of the suture device provided by the embodiment does not need to be provided with two upper fixing holes 1232, the lower shell 141′ does not need to be provided with two lower fixing holes, and the elastic piece 143′ does not need to be provided with fixing holes, so that three corresponding screw holes do not need to be arranged on the second housing 112′.

The “hole” mentioned in the present invention is not necessarily circular and may be square, oval, etc., which are encompassed within the scope of the “hole” to be expressed in the present invention. The terms “first” and “second” as referred to in the present disclosure are merely used to distinguish the elements for convenient nomenclature and do not denote that the first and second must be present at the same time. The terms “upper” and “lower” in the present invention are each opposed to each other and are not intended to be limiting.

To sum up, the suture device, treatment device and treatment system of the present invention can be used for natural orifice transluminal surgery (non-invasive surgery), minimally invasive surgery, or invasive surgery. The suture device is simple in structure and low in manufacturing cost, the advancing of the suture needle can be controlled only through the needle feeding assembly, intermittent suturing or continuous suturing is carried out in soft tissue, the operation is simple, and the risk of surgical failure is greatly reduced. In addition, the suture device of the present invention also includes a needle anti-backoff assembly enabling continuous advancement of the suture needle. In particular, the needle feeding assembly and the needle anti-backoff assembly of the present invention are in a separable engagement relationship with the suture needle, so that complex functions (e.g., suturing of the target suture tissue and knotting of the suture line) can be achieved through a simple structure, the occupied volume of the suture device is small, and the manufacturing cost is low. In the prior art, if the suturing of the tissue in the body is required to be in-vitro operation, the suturing function can be achieved only by matching a spin-drilling and hooking device in the whole process, and the existing spin-drilling and hooking device spins and hooks the tissue directly and then pull it up. The suture device in the present invention does not need to adopt the spin-drilling and hooking device in the suturing process, so that the suturing function can be achieved independently, the damage to the tissue by the spin-drilling and hooking device is avoided, and the surgical risk is greatly reduced.

Those skilled in the art will readily observe that numerous modifications and alterations of the device and method may be made without departing from the spirit and scope of the invention. Therefore, the scope of the appended claims should be accorded the broadest interpretation so as to encompass all such modifications and similar arrangements.

What is claimed is:

1. A suture device, comprising:
a main shell;
a suture needle, wherein one end of the suture needle is connected with a suture line, and the other end of the suture needle is a tip;
a needle feeding assembly disposed on the main shell and forming a separable engagement relationship with the suture needle to control advancement of the suture needle; and
a needle anti-backoff assembly disposed on the main shell and forming a separable engagement relationship with the suture needle to prevent retraction of the suture needle.

2. The suture device of claim 1, wherein the main shell is provided with a sliding groove; the needle feeding assembly comprises a pull cord, an upper shell, a spring, a needle feeding tooth and a sliding block, the pull cord penetrates through the sliding block and the upper shell, the needle feeding tooth is mounted in the sliding block, one end of the spring abuts against a surface of the needle feeding tooth, the other end of the spring abuts against the upper shell, the needle feeding tooth, the sliding block and the spring are mounted in the sliding groove of the main shell, and the upper shell covers the sliding groove.

3. The suture device of claim 1, wherein the main shell has a sliding groove, the needle anti-backoff assembly comprises a needle anti-backoff tooth, a lower shell and an elastic piece, the needle anti-backoff tooth is mounted at the main shell, the needle anti-backoff tooth and the lower shell is mounted in the sliding groove of the main shell, and the lower shell and the elastic piece are fixed to the main shell.

4. The suture device of claim 1, wherein the main shell comprises a first housing and a second housing, the first housing has a connecting slot, the second housing has a connecting projection, and the connecting protrusion is inserted into the connecting slot to connect the first housing and the second housing together.

5. A treatment device for use in conjunction with an endoscope, the treatment device comprising:
a suture device secured to a distal end of the endoscope, the suture device comprising:
a main shell;
a suture needle, wherein one end of the suture needle is connected with a suture line, and the other end of the suture needle is a tip;
a needle feeding assembly disposed on the main shell and forming a separable engagement relationship with the suture needle to control advancement of the suture needle; and
a needle anti-backoff assembly disposed on the main shell and forming a separable engagement relationship with the suture needle to prevent retraction of the suture needle; and
an operating handle secured to an operating end of the endoscope, the operating handle controlling the needle feeding assembly to control the advancement of the suture needle.

6. The treatment device of claim 5, further comprising a connecting ring configured to be sleeved on the endoscope.

7. The treatment device of claim 5, further comprising a snare ring configured to perform a cutting action onto tissue at the distal end of the endoscope.

8. The treatment device of claim 5, further comprising a hook clamp configured to perform a clamping action onto tissue or a hooking action of the suture line at the distal end of the endoscope.

9. A treatment system, comprising:
an endoscope,
a treatment device for use with an endoscope, the treatment device comprising a suture device and an operating handle,
a suture device secured to a distal end of the endoscope, the suture device comprising:
a main shell;
a suture needle, wherein one end of the suture needle is connected with a suture line, and the other end of the suture needle is a tip;
a needle feeding assembly disposed on the main shell and forming a separable engagement relationship with the suture needle to control advancement of the suture needle; and
a needle anti-backoff assembly disposed on the main shell and forming a separable engagement relationship with the suture needle to prevent retraction of the suture needle; and
an operating handle secured to an operating end of the endoscope, the operating handle controlling the needle feeding assembly to control the advancement of the suture needle.

10. The treatment system to claim 9, further comprising a connecting ring, a snare ring and a hook clamp, wherein the connecting ring is configured to be sleeved on the endoscope, the snare ring is configured to perform a cutting action onto tissue at the distal end of the endoscope, and the hook clamp is configured to perform a clamping action onto the tissue and a hook action of the suture line at the distal end of the endoscope.
